(19) United States
(12) Patent Application Publication
(10) Pub. No.: US 2010/0160348 A1
(43) Pub. Date: Jun. 24, 2010

(54) MATERIALS AND METHODS FOR DETECTING AND TREATING PERITONEAL OVARIAN TUMOR DISSEMINATION INVOLVING TISSUE TRANSGLUTAMINASE

(76) Inventor: DANIELA MATEI, INDIANAPOLIS, IN (US)

Correspondence Address: BAKER & DANIELS LLP, 300 NORTH MERIDIAN STREET, SUITE 2700, INDIANAPOLIS, IN, 46204, US

(21) Appl. No.: 12/537,311

(22) Filed: Aug. 7, 2009

Related U.S. Application Data
(63) Continuation of application No. PCT/US08/53320, filed on Feb. 7, 2008.
(60) Provisional application No. 60/888,633, filed on Feb. 7, 2007.

(51) Int. Cl. C12Q 1/52; G01N 33/573; A61K 31/519; C12N 5/09

(52) U.S. Cl. 514/260.1; 435/16; 435/7.4; 514/267; 435/375

(57) ABSTRACT

Tissue transglutaminase-2 (TG2) is involved in Ca⁺⁺-dependent aggregation and polymerization of proteins. TG2 is upregulated in epithelial ovarian cancer (EOC) cells as compared to normal ovarian epithelium. TG2 is also highly expressed in ovarian tumors and secreted in malignant ascites, but it is not detected in non-transformed ovarian epithelium. Furthermore, TG2 enhances EOC cell adhesion to fibronectin and haptotactic cell migration. This phenotype is preserved in-vivo, where the pattern of tumor dissemination in the peritoneal space is dependent on TG2 expression levels. TG2 knock down diminishes dissemination of tumors on the peritoneal surface and mesentery in an i.p. ovarian xenograft model. This phenotype is due to deficient β1 integrin-fibronectin interaction, leading to weaker anchorage of cancer cells to the peritoneal matrix. Highly expressed in ovarian tumors, TG2 facilitates intra-peritoneal tumor dissemination, by enhancing cell adhesion to the extracellular matrix and modulating β1 integrin subunit expression. Accordingly, high levels of TGL activity is implicate in the dissemination ovarian cancer cells. Methods for diagnosing and monitoring ovarian cancers include assessing the level of TG2 and activity. Materials and methods for detecting, preventing and/or treating peritoneal metastasis of ovarian cancers including measuring the level and or activity of TG2 and/or administering compounds that regulate the level and/or activity of TG2.

### PRIORITY CLAIM

This application is a continuation of PCT/US08/53320 filed on Feb. 7, 2008, which is incorporated herein by reference in its entirety, and which claims the benefit of U.S. Provisional Patent Application No. 60/888,633 which was filed on Feb. 7, 2007, and which incorporated herein by reference in its entirety.

### FIELD OF THE INVENTION

Various aspects relate generally to compounds, strategies and methods for diagnosing, preventing and treating peritoneal metastasis of ovarian cancers by detecting changes in the level of tissue transglutaminase (T2G) in ovarian cancer cells and the peritoneal and/or by administering compounds that modulate the level of T2G and its activity.

### BACKGROUND

Ovarian cancer is an idiopathic disease, which affects million of women worldwide. With the exception of Japan, the disease is more common in industrialized nations than it is in developing nations. In the United States, there is about a 1 in 50 chance that a woman will be diagnosed with the disease sometime in her lifetime. Ovarian cancer ranks as the fifth leading cause of cancer related death in women.

Ovarian cancer is characterized by the development of tumors within or associated with the ovaries. Ovarian cancers are characterized by which portion of the ovaries and related tissue the tumors form in, and by the size and morphology of the tumors themselves. Ovarian tumors are classified using the FIGO (Federation Internationale de Gynecolgie et d′Obstetrique) Staging System. This system classifies ovarian tumors in stages through I-IV. Stage I tumors are the most readily treatable; for purposes of prognosis many clinicians group stages II-IV together. Tumor morphologies vary widely and staging tumors is important in determining the best type of therapy to use in treating the tumor. A definitive diagnosis of the disease generally requires histological examination of a sample of tissue collected from the tumor itself. Because of the diverse number of tumor morphologies known to exist, a correct diagnosis generally requires examination by clinicians and pathologists that are well acquainted with ovarian cancer.

The most common treatment for ovarian cancer is surgical removal of the tumor and related tissue. Followed by chemotherapy and/or radiological treatment to destroy any cancer cells that were not removed by the surgery, in many cases additional gynecological tissue is removed to guard against metastasis. In most cases, patients with Types II-IV tumors are treated post surgery with chemotherapy. Regardless of the type of treatment used, the most reliable indicators of a favorable outcome are the patient's age and early diagnosis of the disease. A patient's chances for a favorable outcome drop dramatically if the disease metastasizes.

As with most cancers, successful treatment is intimately tied to early diagnosis. Unfortunately, ovarian cancer is difficult to detect in its early stages and the type of tumors are hard to identify. The vast majority of women later diagnosed with ovarian cancer are asymptomatic during the early stages of the disease. In view of these unfortunate realities ovarian cancer has been dubbed “the silent killer”. Difficulty diagnosing the disease and the dearth of non-surgical treatments for ovarian cancer likely contribute to its lethality. Accordingly, there is a need for new materials and methods for diagnosing and treating ovarian cancer, especially forms that have, or that are likely to, metastasize. Various aspects of the disclosure herein are drawn towards addressing these needs.

### SUMMARY

One embodiment includes a method of assessing patients diagnosed with or at risk for developing ovarian cancer comprising the steps of obtaining a sample of material from an ovarian tumor, ovary or related and surrounding tissues and organs, and analyzing the sample for the presence of elevated levels of Tissue Transglutamise-2 (TG2) activity. Such samples include, but not limited to, samples of tumor tissue, fluids produced by or in contact with the ovaries, fluids or gene products produced by cancer cells associated with the disease, and any related tumors of cancerous tissues or products thereof. Various assays include, but are not limited to antibody based systems for the detection and/or measuring the level of TG2 in at least a portion of the sample collected. Levels of TG2 or TG2 activity are then correlated with an increased likelihood of ovarian cancer or an increased risk for developing ovarian cancer. The sequence of a representative tissue transglutaminase isolated from humans is provided by way of example, and not limitation, as SEQ. ID. NO. 1.

Still another embodiment includes materials and methods for reducing the level or activity of TG2 in patients diagnosed with, or thought to be at risk for developing peritoneal metastasis related to ovarian cancer. In one embodiment the level of TG2 or the activity of TG2 is reduced in order to prevent and/or treat peritoneal metastasis. In still another embodiment peritoneal metastasis due to ovarian cancers is detected, prevented and/or treated by reducing the level and/or activity of TG2. Various embodiments for reducing TG2 levels or activity include, for example, reducing the level of expression, transcription or translation of genes encoding TG2. Yet another embodiment is introduction of a vector, construct, or compound that reduces or inhibits the biosynthesis of at least isoform of TG2. Still other embodiments include, but are not limited to, administering a therapeutically effective dose of at least one agent or compound that inhibits TG2 activity. Yet another embodiment is a method of treating complications of ovarian cancers, such as peritoneal metastasis, by inhibiting or reducing TG2 activity by changing the levels of other cellular proteins, including for example, β1-integin.

One embodiment is a method of diagnosing or according the FIGO classifications ovarian cancer by measuring the level and/or activity of TG2 associated with the tumors.

Still another embodiment includes methods for assessing the likelihood that an ovarian cancer will or has metastasized. In some embodiments these methods comprise the steps of assessing the level of TG2 and/or its activity in at least one of the following; the primary tumor, peritoneum, peritoneal fluid, tissues in the peritoneal space, ascites, ascites fluid and the like. Still another embodiment comprises a method of assessing or diagnosing peritoneal metastasis of ovarian cancer comprising the steps of: obtaining a sample of either tissue or fluid from a patient; and measuring the levels of TG2 or the activity of TG2 in at least a portion of the sample. In one embodiment the sample is drawn from at least one of the following types of tissues or fluids, the primary tumor, peritoneum, peritoneal fluid, tissues in the peritoneal space, ascites, ascetic fluid and the like.

Yet another embodiment includes the step of measuring the interaction between TG2 and β1 integrin to determine is an ovarian cancer has or is likely to metastasize into or past the peritoneal stroma and/or mesothelium.

Still another embodiment is a method of treating ovarian cancer comprising the step of administering to a human or animal patient in need thereof a therapeutically effective dose of at least one compound that alters the activity of TG2. In one embodiment the compound is selected from the group of compounds including cystamine, N-benzyloxy carbonyl, 5-deazo-4-oxonorvaline p-nitrophenylester, glycine methyl ester, CuSO₄, tolbutamide, monodanzyl cadaverine, putrescine, a monoamine, a diamine, gamma-amino benzoic acid, phenylthiourea-(CH₂)ₙ—NH₂, wherein, n is =to 2, 3, 4, 5 and derivates thereof including pharmaceutically acceptable salts and/or formulations thereof.

Still another embodiment is a method of treating ovarian cancer comprising the step of administering to a human or animal patient in need thereof a therapeutically effective dose of at least one compound that alters the activity of TG2 in which the compound is selected from the group of compounds including the compounds of formula 1, 2 and 3, and derivates thereof including pharmaceutically acceptable salts and/or formulations thereof.

Still another embodiment is a method of treating ovarian cancer comprising the step of administering to a human or animal patient in need thereof a therapeutically effective dose of at least one compound that alters the activity of TG2 in which the compound is selected from the group of compounds including the compounds of formula 4, 5 and 6, and derivates thereof including pharmaceutically acceptable salts and/or formulations thereof.

Still another embodiment is a method of treating ovarian cancer comprising the step of administering to a human or animal patient in need thereof a therapeutically effective amount of at least one compound or construct that alters the level of TG2 and/or level of TG2 activity in the patient. In one embodiment the constant is an IRNA directed towards reducing expression of at least one isoform of TG2. In still another embodiment the contract produces anti-sense nucleic acid product that reduces or inhibits the expression of at least one in the form of TG2.

Another embodiment is a method of treating ovarian cancer comprising the step of administering to a human or animal patient in need thereof a therapeutically effective amount of IRNA that reduces the level of TG2 and/or TG2 activity in a patient.

Yet another embodiment is a method of treating ovarian cancer comprising the step of administering to a human or animal patient in need thereof a therapeutically effective amount of an antisense construct that reduces the level of TG2 and/or TG2 activity in a patient. In embodiment the antisense construct is antisense transglutaminase-2 (AS-TG2).

### BRIEF DESCRIPTION OF THE FIGURE

FIG. 1A. Expression of TG2 by IHC in ovarian tumors and normal ovary. Panel A shows photomicrographs of a normal ovary, the arrows point to normal ovarian epithelium.

FIG. 1B. Expression of TG2 by IHC in ovarian tumors and normal ovary. Panel B shows photomicrographs of a normal ovary, the arrows point to a surface epithelial inclusion.

FIG. 1C. Expression of TG2 by IHC in ovarian tumors and normal ovary. Panel C shows photomicrographs of representative ovarian tumors.

FIG. 1D. Expression of TG2 by IHC in ovarian tumors and normal ovary. Panel D shows photomicrographs of representative ovarian tumors

FIG. 2. Images of immunoblots showing TG2 expression in ascites specimens, each immunoblot includes 6 specimens of malignant ascites collected from patients with EOC (lanes 1-6), 2 specimens of ascites fluid or pleural fluid from patients with non-malignant diseases (lanes 7-8) and a positive control (lysate from ovarian cancer cell line, SKOV₃). Equal volume (30 μl) of ascites fluid was loaded in each lane of the gel.

FIG. 3A. Images of immunoblots showing level of TG2 and GAPH in samples. Data illustrating the effects of TG2 knock-down treatments on ovarian cancer cell adhesion and migration. Panel A, shows the results of a Western blot assay for TG2 using, sample from cell lysates and conditioned media (CM). Lanes labeled clone G and clone M show stable clones identified by selection with G418 after transfection with AS-TG2. Controls are samples from un-transfected cells (UT) and cells that were stably transfected with pcDNA3.1. Serum free CM from AS-TG2 clone (G) was compared to CM from pcDNA3.1 transfected cells were detected by immunoblotting. Equal volumes of CM were loaded in each lane (30 μl).

FIG. 3B. B shows solid phase assay results obtained by measuring adhesion to FN for vector and AS-TG2 transfected cells. The number of cells adhering to FN within one hour was quantified based on fluorescence emission after cells were labeled with Calcein AM relative fluorescence units (RFU). Data points represent means of 4 replicates +/− SE; p-value=0.006.

FIG. 3C. Images stained for phalloidin in SKOV3 cells stably transfected with AS-TG2 or with vector. Cells were plated on FN coated cover slips, allowed to adhere for 60 minutes, then fixed and stained with rhodamine-phalloidin antibody. Visualization was performed under UV excitation at 520 nm with a confocal microscope (red signal).

FIG. 3D. Bar graph summarizing data showing the effect of AS-TG2 and pCDNA on collagen or FN measured in SKOV3 cells stably transfected with AS-TG2 or with pCDNA3.1, about 1×10⁶ cells were plated in each well. Cells migrating to the lower surface of the filter within 5 hours were counted. Values represent averages of cells counted per 10 HPF for each experimental condition +/−SE (p-value<0.001).

FIG. 3E. Bar graph summarizing data collected on the directional migration stimulated by fibronectim (FN) and conditioned media, measured in SKOV3 cells, about. 1×10⁶ SKOV3 cells were plated in each well. In the lower chamber the media consisted of: serum free media (1), media with 20% FBS (2), serum free CM collected from SKOV3 cells stably transfected with AS-TG2 (3) or with pcDNA3.1 (4). Cells migrating to the lower surface of the filter within 5 hours were counted. Values represent averages of cells counted per 10 HPF +/−SE (p-value<0.001).

FIG. 4A. Effects of TG2 over expression on ovarian cancer cell adhesion and directional migration. Images of a Western blot analysis of TG2: OV90 cells transfected with TG2 or pcDNA3.1 vector were selected with G418. A Western blot assay for TG2 identifies clone #17 as a stable clone expressing TG2.

FIG. 4B. Graph summarizing the results of a solid phase assay measuring adhesion to FN for cells stably transfected with TG2 or with pcDNA3.1. Data points represent means of 4 replicates +/−SE (p-value=0.01).

FIG. 4C. Bar graph illustrating the effect of collagen or Fibromatic (FN) on the migration of cells in OV90 cells stably transfected with TG2 or pcDNA3.1, 1×10⁶ cells were plated in each well. Cells migrating to the lower surface of the filter within 5 hours were counted. Values represent averages of cells counted per 10 HPF for +/−SE (p-value<0.00001).

FIG. 5A. The images of open animals exposing macroscopically the small bowel. TG2 knock-down inhibits tumor development and i.p. dissemination in-vivo. A) In-vivo tumor development. Nude mice injected with control cells (pcDNA3.1-SKOV3) form tumors studding the mesentery and peritoneal surface, as well as tumors infiltrating the RP space. Mice injected with AS-TG2 transfected cells form tumors in the RP space, at the site of the i.p. injection, and have clear mesentery. In the animal injected with pcDNA3.1 transfected cells, an arrow points to large tumors on the mesentery, adjacent to the small bowel. In the mouse injected with AS-TG2 cells, an arrow points to a tumor nodule at the site of i.p injection. Macroscopically, the bowel and mesentery appear clear. Pieces of small bowel and adjacent mesentery were photographed at 12× magnification with a StemiSV11 ApoZeiss dissecting microscope. In the mouse injected with AS-TG2 cells, the arrow points to clear mesentery. In the animal injected with control cells, the multiple arrows indicate many tumor implants, studding the mesentery.

FIG. 5B. Images showing the histological appearance of xenografts (hematoxillin and eosin staining): 1) no tumor is visualized on the mesentery in animals injected with AS-TG2 cells; 3) block of tumor derived from pcDNA3.1-SKOV3 cells invading the mesentery, adjacent to bowel; 2 and 4) AS-TG2 and pcDNA3.1 derived tumor infiltrating the pancreas. Arrows point to tumor deposits in sections 2, 3, and 4, respectively. In section 1, the arrow points to clear mesentery, adjacent to normal bowel.

FIG. 5C. Images illustrating the effect of expressions of TG2 by IHC in xenografts: Section (1) is a negative control (no primary antibody, pcDNA3.1 derived tumor), TG2 staining is absent in tumors derived from AS-TG2 cells; Section (2) shows intense TG2 staining noted in tumors derived from pcDNA3.1 transfected SKOV3 cells Sections (3 and 4). Section (3) depicts a 3+TG2 peritoneal implant in the mesentery, adjacent to normal bowel (arrow).

FIG. 5D. Images of immunoblots designed to detect for TG2 and β1 integrin in lysates from xenografts-derived cell cultures: the first lane is pcDNA3.1 xenograft derived culture; and the second lane represents a culture established from an AS-TG2 derived xenograft.

FIG. 5E. A graph illustrating decreased adhesion to FN in cells derived from pcDNA3.1 and AS-TG2 xenografts. Adhesion to FN was measured by solid phase assays for cells cultured from explanted xenografts. The graph depicts the fold difference in measured fluorescence (RFU) corresponding to the number of cells adherent within 1 hour to FN-coated surfaces, FN concentrations varied between 1-10 μg/mL.

FIG. 6A. Interaction between TG2 and β1 integrin. An image showing the results of immunoprecipitation (IP) assays carried out with monoclonal anti-β1 integrin antibody or IgG and Western blot analysis using anti-TG2 antibody. Input represents 50 μg of cell lysate.

FIG. 6B. Photographs of an immunofluoresence assay carried out with polyclonal anti-TG2 antibody (secondary antibody labeled with AlexaFluor⁴⁸⁸, green) and a monoclonal anti-β₁ integrin antibody (secondary antibody labeled with Cy5™, red) used to identify cellular localization of the two proteins. Protein co-localization is identified by emergence of yellow spectra (large arrow) on merged images and was quantified by using the Metamorph software in a Z-stack of images. 64% of β1 integrin co-localized with TG2. Nuclei were visualized by DAPI staining.

FIG. 7A. Down regulation of TG2 in ovarian cancer cells correlates with decreased β1 integrin expression and presentation to the cell membrane. Image showing the results of Western blot analysis for TG2 and β1 integrin in SKOV3 cells stably transfected with eitherpcDNA3.1 or AS-TG2.

FIG. 7B. Image showing the results of RT-PCR for TG2 and β1 integrin in cells stably transfected with either pcDNA3.1 or AS-TG2.

FIG. 7C. Graphic summary of analyzing cells stably transfected with either pcDNA3.1 or AS-TG2 to measure the effect of β1 integrin expression.

FIG. 7D. Image showing the results of Immuno-flourescent staining for β1 integrin (secondary antibody labeled with AlexaFluor⁴⁸⁸, green) in cells stably transfected with pcDNA3.1 or AS-TG2.

FIG. 7E. Image showing the results of Immunoblotting designed to detect β1 integrin in the membrane and cytosolic fractions of cells stably transfected with pcDNA3.1 or AS-TG2. Blotting for EGFR levels was used as a control to identify the membrane fraction.

FIG. 8: Table 1, includes data illustrating the relationship between ovarian cancer tumor formation and the expression of TG2.

### DESCRIPTION

For the purposes of promoting an understanding of the principles of the invention, reference will now be made to the preferred embodiments thereof, and specific language will be used to describe the same. It will nevertheless be understood that no limitation of the scope of the invention is thereby intended, such alterations, modifications, and further applications of the principles of the invention being contemplated as would normally occur to one skilled in the art to which the invention relates.

A number of explanations and experiments are provided by way of explanation and not limitation. No theory of how the invention operates is to be considered limiting whether proffered by virtue of description, comparison, explanation or example. Accordingly, the following examples and discussion are presented by way of guidance and explanation and not limitation.

The compositions and methodologies disclosed and implied herein, are useful in both humans and other animal (e.g. pets, zoo, or domestic animals) applications.

Unless noted otherwise, the term “treating,” as used herein includes curing, controlling, inhibiting, slowing the progression of, and/or preventing the advance of a disease. For example, one aspect includes treating ovarian cancer using materials and/or methods that prevent or slow the advance of the disease from one stage to the next and/or preventing or slowing the dissemination of epithelial ovarian cancer cells, and helping to control, slow or prevent cancerous cells from spreading to, onto and/or beyond the peritoneal surface and mesentery.

Epithelial ovarian cancer (EOC) arises from the epithelial layer covering the surface of ovaries and intra-peritoneal (i.p.) metastasis of the disease is commonly observed at diagnosis. Ovarian tumor spread in the i.p. space leads to the characteristic symptoms and complications of the disease, ascites and small bowel obstruction. Several features set apart the spread of ovarian cancer from the metastatic model characteristic of other epithelial tumors. First, EOC cells are in direct contact with the overlying peritoneal fluid and this allows exfoliated cells to disseminate freely in the i.p. space. Second, ovarian cancer cells derived from the mullerian epithelium have dual epithelial and mesenchymal characteristics and can convert to either phenotype in response to factors in the micro-environment. Adopting a mesenchymal phenotype favors dislodgement from the primary tumor, as mesenchymol cells are more motile and not bound by tight cellular junctions. Thus EOC cells can spread passively to distant sites by exfoliating from the primary tumor, floating in the peritoneal fluid and nesting along the i.p. space, where they adhere and grow as metastatic implants. This type of spread which is uniquely characteristic to EOC, is accompanied by specific changes at the interface between tumor and the peritoneal “oncomatrix” that allow these cancer cells to move, attach and grow.

Such changes include increased expression of integrins and of the hyuloran receptor CD44 that promote adhesion of EOC cells to the peritoneum; over expression of the chemokine receptor CXCR4 and secretion of its ligand CXCL12 that regulate their motility in the i.p. milieu. Cancer and mesothelial cells secrete lypophosphatidic acid and other proteins (fibronectin, periostin, osteopontin, laminin, which stabilize the ECM and promote establishment of metastases. These interactions with the mesothelium and the peritoneal stroma activate “outside-in” signaling which stimulates cancer cell proliferation and survival. In this context, neovascularization is facilitated and peritoneal metastases form and grow.

Human tissue transglutaminase (TG2), e.g. SEQ ID 1 amino acids, is a ubiquitously expressed enzyme, involved in protein cross-linking via acyl-transfer between glutamine and lysine residues. TG2 promotes Ca⁺⁺-dependent post-translational protein modification effected by insertion of isopeptide bonds and incorporation of polyamines into peptide chains. TG2 is found widely in nature and various forms of the protein isolated from different organisms have been found to include several conserved features.

TG2 mRNA expression is up-regulated in transformed ovarian epithelial cells and tumors compared to normal ovarian surface epithelial cells. TG2 is also linked to epithelial cancers, particularly pancreatic, breast and non-small cell lung cancer. In breast cancer cells, membrane-bound TG2 has kinase activity and phosphorylates the insulin-growth factor binding protein-3 (IGFBP-3). This protein is over-expressed in drug and radiation-resistant breast cancer cells. As disclosed herein TG2, serves as a mediator of ovarian tumor dissemination in the peritoneal space.

TG2 is expressed in a cancer-specific manner in human ovarian tumors and it is secreted into ascites fluid. As disclosed herein, TG2 facilitates ovarian cancer cell adhesion to fibronectin (FN), haptotactic cell migration and promotes intraperitoneal tumor seeding. TG2 may exert its role by interacting with β1 integrin, modulating its expression and function. This suggests a novel role for TG2, placing it at the interface between tumor cells and the peritoneum, as a regulator of i.p. metastasis. For additional discussion please see Satpathy, et al., Cancer Res. 2007 67(15); 7194-202.

Intraperitoneal metastasis characteristic to EOC requires modifications of tumor cells to facilitate interaction with the peritoneal stroma and mesothelium. The over-expression of TG2 in ovarian tumors and its secretion into malignant ascites likely has a role in the metasis of these cells. Data disclosed herein shows that TG2 appears to mediate ovarian cancer cell adhesion to FN and stimulates directional cell motility, these processes being mediated by TG2 via interaction and stabilization of β1 integrin. Data collected using an i.p. xenograft model, indicates that TG2 knock-down decreases the pattern of diffuse tumor spread, implicating it as a mediator of i.p. metastasis.

As disclosed herein, TG2 is surprisingly over-expressed in primary EOC cells. Data disclosed herein indicate that >80% of ovarian tumors over-express transcripts of TG2. Surprisingly, TG2 is not expressed in the surface ovarian epithelium, but it is present in stage I and II ovarian tumors, illustrating that its up-regulation is an early event in EOC. TG2 up-regulation has been reported in glioblastoma, pancreatic, breast and in lung cancer and a multitude of functions have been invoked for it.

We report here on TG2's induced stabilization of cell adhesion, as this appears to be critical to the establishment of i.p. metastases in ovarian cancers, where cancer cells are required to “stick” to the onco-matrix in order to establish peritoneal implants. As disclosed herein, adhesion to FN and chemotaxis were decreased in EOC cells by knock down of TG2 and enhanced by stable over-expression of TG2. This phenotype was preserved in-vivo, where the pattern of distribution of i.p. implants was remarkably altered by TG2 knock-down. Control animals injected with SKOV3-pcDNA3.1, a construct that express TG2 cells, developed AS-TG2implants widely disseminated on the mesentery and the peritoneal/hepatic gutters. Animals injected with SKOV3-AS-TG2 cells (a construct that reduces the level of TG2) developed large tumors in the retroperitoneal space and few mesenteric metastatic foci, these results suggest that TG2 plays an important role in mediating the development of peritoneal metastasis. The volume of dominant masses was equal or slightly higher in animals injected with AS-TG2 cells than in controls, whereas peritoneal seeding was decreased. These observations are consistent with a potential dual role for TG2 in tumor development. TG2 may act as a negative regulator of primary tumor growth, as observed in melanoma. As disclosed herein, we demonstrate that in EOC, TG2 promotes tumor spread, possibly by enhancing adhesion of cancer cells freely floating in the peritoneal fluid to the mesothelium and the ECM.

Without being bound by any specific theory or explanation, the altered metastatic phenotype of ovarian cancers observed herein is likely due to deficient interaction between tumor cells and the extracellular matrix in the absence of TG2. TG2 in complex with the β1 integrin may enhance adhesion to FN in fibroblasts by binding to its gelatin domain. This is unexpected in view of reports that, TG2 and β1 integrin do not interact in vitro. Our unpublished observations also show that addition of exogenous TG2 (recombinant TG2 or protein purified from guinea pig liver) fails to increase ovarian cancer cell adhesion to FN, suggesting that other factors may also modulate the TG2-integrin-FN interaction. Indeed, we found diminished expression of the β1 subunit on the surface of cells where TG2 was stably down-regulated.

Again, without being bound by any theory or specific explanation, we propose that TG2 is required for β1 integrin processing as it is presented to the cell membrane. As β1 can complex with several α subunits, modulating cell-matrix interactions, we suggest that TG2 down regulation may affect such integrin complex formation, leading to deficient cell adhesion to the ECM. These unexpected results highlight a novel function for transglutaminase, distinct from its previously suggested role of “co-receptor” for FN. By directly modulating integrin expression levels, TG2 may impact invasiveness of ovarian cancer, clinical outcome and, potentially the sensitivity of these types of cancers to various forms of chemotherapy.

In light of these results, one embodiment is materials and methods for detecting and diagnosing ovarian cancer. In one embodiment this can be accomplished by monitoring the level of TG2 and TG2 activity in patients with, or at risk for, developing ovarian cancer. Still, another aspect includes materials and methods for treating or preventing peritoneal metastasis associated with ovarian cancers by regulating the level and/or activity of TG2 in cells associated with ovarian cancer, including, for example altering levels of TG2 in the cells or its interactions with other proteins.

As disclosed herein, we determined that TG2 is secreted abundantly in malignant ascites. Again what being bound by any theory, or explanation, this may be due directly to the accumulation of TG2 produced by tumor and/or mesothelial cells, as primary ovarian cancer cell lines secrete TG2. Although, TG2 lacks a leader sequence, it appears to be secreted in the extracellular space, through a yet unknown mechanism. The detection of TG2 in ascites indicates that TG2's expression in tumor cells and its presence there may remodel the “oncomatrix” perhaps by helping to cross-link ECM proteins. Other factors thought to be critical to i.p. metastasis, and reported to be abundantly secreted by ascites include, but are not limited to, FN, lysophosphatidic acid, hyaluran, and VEGF, making the i.p. milieu favorable for cancer cell growth.

One unexpected result reported herein is that TG2 is highly expressed in human ovarian tumor cells and secreted in malignant ascites. At the interface between cancer cells and the stroma, TG2 appear to alter the pattern of tumor growth and dissemination in the peritoneal space, perhaps by modulating β1 integrin expression and function, thoroughly supporting and/or promoting peritoneal metastasis in ovarian cancer. Blocking, or at least reducing the expression of TG2 in ovarian cancer cells or model for its disease, reduces the tendency of it to metastasize. Accordingly, blocking production of TG2 or TG2 activity may reduce the likelihood that ovarian cancer will metastasize. These results will likely have a direct therapeutic effect on patients with ovarian cancer.

### EXAMPLES

### Materials and Methods

Immunohistochemistry: Twenty seven paraffin-embedded epithelial ovarian tumor specimens from the Cooperative Human Tissue Collection (CHTN), and six normal ovarian specimens from six patients undergoing oophorectomy for benign disorders from the Indiana University Tissue Bank Collection, were immunostained using a TG2 monoclonal antibody (CUB 7402, Neomarkers, Fremont, Calif.) at a dilution of 1:200 after antigen retrieval using sodium citrate. Secondary labeling was based on the Avidin/Biotin system (Dako, LSAB2 kit). Slides were stained with 3-3′ diaminobenzidine (DAB) and counterstained with hematoxyllin. Negative controls were run in parallel, with omission of the primary antibody. Staining was graded from 0 (no staining) to 3+ (strong staining) by a board certified pathologist. Immunoreactivity was recorded only if noted in more than 15-20% of tumor cells. The Indiana University Institutional Review Board approved the use of human tissue specimens (protocol# 0412-54).

Ascites fluid: Thirty samples of ascites fluid cytologically positive from patients with ovarian cancer and eight samples of ascites fluid from patients with non-cancerous conditions (inflammatory pleural or ascitic fluid) were included in this analysis (UCLA and NCC Tissue Bank, protocol collection approved by the Institutional Review Board, protocol #0409-02). After collection, samples were centrifuged to remove cellular debris, aliquoted and stored at −80° C. until use.

Cell lines: Human SKOV₃ and OV90 ovarian cancer cell lines were obtained from the American Type Culture Collection (ATCC, Manassas, Va.) and cultured in growth media containing 1:1 MCDB 105 (Sigma, St Louis, Mo.) and M199 (Cellgro, Herndon, Va.) supplemented with 10% heat inactivated fetal bovine serum (FBS, Cellgro) and 1% antibiotics (100 units/mL penicillin and 100 μg/mL streptomycin). All cells were grown at 37° C. in a humidified 5% CO₂ atmosphere.

Transfection: To over-express TG2, OV90 cells in the logarithmic phase of growth were transfected with wild type (wt) TG2 cloned into the pcDNA3.1 vector (Invitrogen, Carlsbad, Calif.) using Fugene (Roche Applied Science, Indianapolis, Ind.). To knock-down TG2, an anti-sense construct cloned in to pcDNA3.1 vector was transfected in SKOV3 cells. As a control, cells were transfected with the pcDNA3.1 vector, carrying the G418 resistance gene. Transfection efficiency in these conditions is typically 5-10% in OV90 cells and 30-40% in SKOV3 cells, as determined by estimation of green fluorescent protein (GFP) expression. Stable transfected clones were established by selection with G418 (Sigma) at concentrations of 600 μg/mL for SKOV3 cells and 150 μg/mL for OV90 cells. Plasmids were generous gift from Professor Janusz Tucholski, University of Alabama. For additional information, on these plasmids please see, for example, Tucholski, et al., Neuroscience, Vol. 102, No. 2, pp. 481-491 (2001). Over-expression and knock-down of TG2 in selected clones was demonstrated by Western Blot analysis.

Conditioned media: Serum free conditioned media was collected from SKOV₃ cells stably transfected with vector (pcDNA3.1) or the TG2 antisense construct (AS-TG2) and centrifuged at 3000 rpm for 5 minutes to sediment cellular debris. Equal volumes of conditioned media (30 μL) were used for immunoblotting.

TSTG2 antisense vector constructs. The antisense vector TSTG2 was a generous gift from Dr. Professor Janusz Tucholski, University of Alabama. Briefly, an antisense vector pcDNA3.1-anti-tTG (AS-TG2) was constructed by subcloning an Eco-R1-Xba1 fragment of tTGcDNA from pcDNA-tTG into pc DNA3.1(−). The construct included 592 by of protein-coding sequence and 135 by of 5″ untranslated sequence of tTG cDNA. For additional information on this vector please see, Tucholski, et al., Neuroscience, Vol. 102, No. 2, pp. 481-491 (2001).

Western blot analysis: Cells were lysed in RIPA buffer containing protease inhibitors leupeptin (1 μg/mL), aprotinin (1 μg/mL) and phenylmethylsulphonyl fluoride (PMSF, 400 μM), and sodium orthovanadate (Na₃VO₄, 1 mM). Cell lysates were sonicated briefly and subjected to centrifugation at 14000 rpm for 15 minutes at 4° C. to sediment particulate material. Equal amounts of protein (50 μg) were separated by SDS-PAGE and electro-blotted onto PVDF membranes (Millipore, Bedford, Mass.). After blocking, membranes were probed with primary antibody overnight at 4° C. with gentle rocking. Antibodies used are β1 integrin antibody (MAB2251, Chemicon, Temecula, Calif., 1:1000 dilution), TG2 (CUB 7402, Neomarkers, Fremont, Calif., 1:1000 dilution), GAPDH (Biodesign International, Saco, Me., 1:5000 dilution) and EGFR (Cell Signaling, Boston, Mass., 1:1000 dilution). After incubation with specific HRP-conjugated secondary antibody, antigen-antibody complexes were visualized using the enhanced chemiluminescence detection system (Amersham Biosciences). Images were captured by a Luminescent Image Analyzer with a CCD camera (LAS 3000, Fuji Film, Stanford, Conn.).

Separation of membrane and cytosolic proteins: SKOV₃ cells were collected in a hypotonic lysis buffer containing 10 mM KCl, 1.5 mM MgCl2, 10 mM Tris-HCl, pH 7.4 and 2 mM PMSF. The cell lysate was centrifuged at 4000×g for 15 minutes to remove cell debris and nuclei. The supernatant was then centrifuged at 100,000×g for 60 minutes to separate the membrane fraction. The final crude membrane pellet was resuspended in a buffer containing 0.25 M sucrose, 10 mM Tris-HCl, pH 7.4, 150 mM NaCl, and 2 mM PMSF.

Co-immunoprecipitation: To detect the interaction between TG2 and β1 integrin, SKOV₃ cells were plated on fibronectin-coated (5 μg/mL) dishes, allowed to adhere for 2 hours and then lysed in a buffer containing 20 mM Tris (pH 7.4), 150 mM NaCl, 1 mM β-glycerolphosphate, 1 mM EDTA, 1 mM EGTA, 1 mM Na₃VO₄, 2.5 mM sodium pyrophosphate, 1% Triton X-100, 10% glycerol, 1 μg/mL leupeptin, 1 μg/mL aprotinin, 400

PMSF and 1 mM DTT. After 30 minutes of incubation on ice, cells were scraped from the plates, sonicated and centrifuged at 14000 rpm for 15 minutes to pellet cellular debris. 500 μg of protein from the supernatant was incubated overnight at 4° C. with β1 integrin monoclonal antibody (Chemicon Temecula, Calif.) or mouse IgG (Santa Cruz Biotechnology, Santa Cruz, Calif.). Immune complexes were recovered by adding 60 μL of a slurry of protein G plus/protein A agarose (Calbiochem, San Diego, Calif.) and shaking for 2 hours at 4° C. After washing with a buffer containing 0.2% Triton X-100, the immune complexes were eluted with 2× sample buffer and boiled for 10 minutes at 100° C.

RT-PCR: RNA was extracted from SKOV3 cells stably transfected with AS-TG2 or empty vector using RNA STAT 60 reagent (Tel-Test Inc, Friendswood, Tex.). 2.5 mg RNA was utilized for first strand cDNA synthesis using the Superscript II system (Invitrogen, Carlsbad, Calif.). The following primers were used: β1 integrin forward (F): ATC TGC GAG TGT GGT GTC TG SEQ. ID No. 2 and reverse (R): ACA ACA TGA ACC ATG ACC TC SEQ ID No. 3 and GAPDH GAT TCC ACC CAT GGC AAA TTC C (F) SEQ ID No. 4 and CAC GTT GGC AGT GGG GAC (R) SEQ ID No. 5 (Integrated DNA Technologies, Coralville, Iowa). The RT product (1 μl) and primers were heated at 94° C. for 90 sec, followed by 28 rounds of amplification for GAPDH and 34 cycles for β1 integrin (30 sec denaturing at 94° C., 30 sec annealing at 60° C. and 30 sec extension at 72° C.), followed by a final extension of 10 minutes at 72° C. The RT-PCR product was visualized under UV light after fractionation on a 1.5% agarose gel.

Immunofluorescence: SKOV₃ cells were plated on fibronectin coated chamber slides (BD Biosciences, Bedford, Mass.) and allowed to adhere. After fixation in 4% para-formaldehyde, cells were permeabilized using Triton X-100 (0.2% in PBS; 15 minutes) and blocked for 1 hour with 3% goat serum in PBS. Then, cells were incubated for 2 hours with primary antibody diluted in blocking buffer at room temperature (TG2 polyclonal antibody RB-060, Neomarkers, 1:100 dilution and β1 integrin monoclonal antibody, MAB2251, Chemicon, 1:100 dilution), followed by a 30 minute incubation with AlexaFluor⁴⁸⁸ anti-mouse secondary antibody (1:1000, Molecular Probes, Eugene, Oreg.) or CY5™-conjugated anti-rabbit antibody (1:500, Zymed, San Francisco, Calif.). Staining to visualize the cytoskeleton was performed with rhodamine-phalloidin (Molecular Probes). Isotype specific IgG served as a negative control. Nuclei were visualized by DAPI staining (Vectashield, Vector Laboratories, Burlingame, Calif.). Analysis was performed using a Zeiss LSM510 meta-confocal multi-photon microscope system under UV excitation at 488 nm (for AlexaFluor⁴⁸⁸), 630 nm (for CY5), 540 nm (for rhodamine) and 340 nm (for DAPI). Protein co-localization was estimated by calculating the area of color overlap in a Z-stack of images using Metamorph software.

Solid phase adhesion assays: Exponentially growing cells were detached from culture plates by trypsinization, and labeled with calcein acetoxymethylester (Calcein AM, 2 μM, Molecular Probes) for 20 minutes. After washing, cells were resuspended in serum-free media. Equal numbers of cells (4×10⁴ cells per well) were seeded into 96 well plates pre-coated with fibronectin (Sigma, St. Louis, Mo.) at different concentrations (1-10 μg/mL) or BSA (1% w/v). After one hour of incubation at 37° C., the plate was immersed into PBS containing 1 mM MgCl₂ to remove non-adherent cells. The number of adherent cells was measured in a fluorescence plate reader (Applied Biosystems, Foster City, Calif.) at an excitation wavelength of 485 nm and an emission wavelength of 530 nm. All experiments were performed in quadruplicate and repeated twice.

Cell migration assays: A migration assay was performed in a modified Boyden chamber method using 6.5 mm diameter, 8.0 μm pore size polycarbonate membrane transwell inserts in a 24 well plate (Corning, N.Y., NY). To assess directional migration, the lower surfaces of the transwell membrane were coated with 50 μg/ml FN (Sigma) or with 0.01% type I collagen (Sigma). SKOV3 cells stably transfected with AS-TG2 or with vector were serum starved for 18 hours and then plated in the upper well at a concentration of 2×10⁵ in 100 μl of serum free media. After 4 hours of incubation at 37° C. in a CO₂ incubator, the cells on the upper surface of the membrane were wiped off with a cotton swab. The cells on the lower surface of the transwell were stained with HEMA3 stain (Fisher), and counted at 200× magnification. Cells were counted in five high power fields in duplicate experiments. Results are expressed as mean number of migrating cells +/−standard error. A similar experiment was performed using serum free CM from cells stably transfected with AS-TG2 or pcDNA3.1 as cell attractant in the lower chamber of the transwell.

In vivo growth of SKOV3 cells in nude mice: The human ovarian cancer cell line SKOV3 stably transfected with AS-TG2 or vector was injected i.p. into 7-8 weeks old female nude mice (nu/nu Balbc) from Harlan, Indianapolis, Ind. Eight weeks after the injection, the mice were euthanized and a necropsy was performed. Tumor formation was estimated by two methods. First, we measured bi-dimensionally tumors >0.4 cm with calipers and calculated tumor volume according to the formula L*W²/2; where L is length and W is width. A cumulative tumor volume was calculated by adding the volumes of dominant tumors for each animal. Second, we estimated peritoneal seeding with un-measurable 1-3 mm tumors, by counting the number of implants on the mesentery, omentum and peritoneum. Harvested tumors were preserved in formalin for future histological and IHC studies. When possible, tumors were minced and cultured in media supplemented with G418 in the presence of hyaluronidase at a concentration of 1 mg/mL. The xenograft-derived cultures were characterized by immunoblotting and solid phase assay. Animal experiments were approved by the Indiana University School of Medicine Animal Care and Use Committee (protocol# 2680) and were in accordance with federal regulations. Three independent experiments were performed.

Flow Cytometry: Quantification of cell surface β1 integrin was performed using the FACScan/CellQuest system (Becton-Dickinson, San Jose, Calif.). Trypsinized cells were incubated with β1 integrin monoclonal antibody (1:100, dilution) or mouse IgG (Santa Cruz Biotechnology) for 1 hour on ice. After incubation with secondary AlexaFluor⁴⁸⁸ labeled anti-mouse IgG (1:500, dilution), immunofluorescent staining was quantified using the FACScan/CellQuest system. Ten thousands events were accumulated for each analysis. Three independent readings were obtained from separate experiments and data were averaged for statistical analysis.

Statistical analysis: For the analysis of the IHC and immunoblotting data in cancer and non-cancer specimens, the chi-square test was utilized. Likewise, the chi-square was used for the comparison between animals developing peritoneal studding in the groups injected with AS-TG2 transfected or with control cells. For the solid phase adhesion and migration assays, the flow cytometry analysis and the comparison of volumes and number of peritoneal implants between the two animal groups, we used the Student t-test.

### Tissues and IHC

Tumor specimens from the Cooperative Human Tissue Collection (CHTN) and the Indiana University (IU) Tissue Bank were immunostained using a TG2 monoclonal antibody (CUB 7402, Neomarkers). Staining was graded from 0 to 3+ by a board certified pathologist. Thirty samples of ascites fluid from patients with EOC and eight samples of ascites fluid from patients with non-malignant conditions (inflammatory pleural or ascites fluid) were included in this analysis (UCLA and IU Tissue Banks). The IRB approved the use of human tissue specimens.

### Cell Lines and Transfection

Human SKOV₃ and OV90 ovarian cancer cell lines, from the American Type Culture Collection (ATCC) were cultured in 1:1 MCDB 105 and M199 supplemented with 10% FBS. To over-express TG2, OV90 cells in the logarithmic phase of growth were transfected with TG2 cloned into the pcDNA3.1 vector. To knock-down TG2, an anti-sense construct (AS-TG2) cloned into pcDNA3.1 was transfected in SKOV3 cells. As a control, cells were transfected with pcDNA3.1 vector. Stable clones were established by selection with G418. Plasmids were from Dr. J. Tucholsky.

### Separation of Membrane and Cytosolic Protein

SKOV₃ cells were collected in a hypotonic lysis buffer containing 10 mM KCl, 1.5 mM MgCl2 and 10 mM Tris-HCl. The lysate was centrifuged at 4000×g for 15 minutes to remove debris and nuclei. The supernatant was then centrifuged at 100,000×g for 60 minutes to separate the membrane fraction. The crude membrane pellet was re-suspended in 0.25 M sucrose, 10 mM Tris-HCl and 150 mM NaCl.

### RT-PCR

The used primers were: β1 integrin forward (F)

SEQ ID No. 3

ATC TGC GAG TGT GGT GTC TG
and
reverse (R)

SEQ ID No. 4

ACA ACA TGA ACC ATG ACC TC
and
GAPDH

SEQ ID No. 5

GAT TCC ACC CAT GGC AAA TTC C (F)
and

(SEQ ID No. 6

CAC GTT GGC AGT GGG GAC (R).

### Immunofluorescence (IF)

SKOV₃ cells were plated on fibronectin coated slides, fixed, permeabilized with Triton X-100 and incubated with primary and fluorescent labeled secondary antibodies. Nuclei were visualized by DAPI staining. Analysis was performed using a Zeiss LSM510 meta-confocal microscope system. Protein co-localization was estimated by calculating the area of color overlap in a Z-stack of images using Metamorph software.

### Solid Phase Adhesion

Equal numbers of cells labeled with calcein acetoxymethylester were seeded into 96 well plates coated with FN. Cells were allowed to adhere for 1 hour and the number of adherent cells was measured in a fluorescence plate reader. All experiments were performed in quadruplicate and repeated twice.

### Cell Migration

Migration assays were performed in modified Boyden chamber method using 8.0 μm pore size polycarbonate membrane transwell inserts. To assess directional migration, the lower surfaces of the transwells were coated with 50 μg/ml FN or 0.01% type I collagen. Cells migrating to the lower surface of the inserts were counted at 200× magnification.

### Growth of SKOV3 Cells in Nude Mice

SKOV3 cells stably transfected with AS-TG2 or vector were injected i.p. into 7-8 week old female nude mice (nu/nu Balbc). Eight weeks after the injection, the mice were euthanized and a necropsy was performed. Two independent experiments were performed and are summarized in Table 1. Tumor formation was estimated by two methods. First, tumors >0.4 cm were measured bi-dimensionally and tumor volume was calculated according to the formula L*W²/2; where L is length and W is width. For each animal a cumulative volume was calculated by adding individual tumor volumes. Second, peritoneal seeding was estimated by counting the number of implants on mesentery, omentum and peritoneum. When possible, tumors were minced and plated to establish xenograft-derived cultures. Animal experiments were approved by the IU Animal Care and Use Committee, being in accordance with federal regulations.

### TG2 is Expressed in EOC

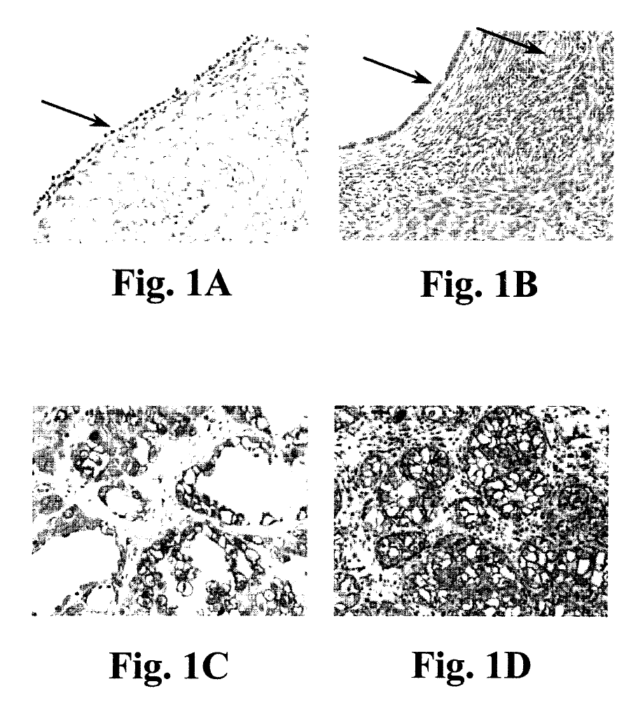

In order to determine the expression levels of TG2 in ovarian tumors, we used immunohistochemisty (IHC). Among 28 tumors, we identified intense cytoplasmic and membrane staining (2-3+ in more than 50% of tumor cells) in 24 specimens (85% of tumors, FIG. 1). Two specimens stained weakly (1+) and four tumors did not stain. All histological subtypes were immunoreactive: 9 of 9 clear cell carcinoma were intensely positive, 3 of 3 endometrioid tumors displayed 2+ staining, one carcinosarcoma specimen was 3+ immunoreactive and 9 of 14 papillary serous tumors were 2-3+ positive. One poorly differentiated carcinoma did not immunoreact. TG2 expression was noted in advanced tumors (10 of 14 stage III and IV tumors), as well as in early stage disease (13 of 14 tumors stage I and II). In normal ovary, TG2 immuno-reactivity was weak in normal stroma and absent in the surface epithelial layer. None of six normal specimens immunostained for TG2 in the epithelium, suggesting that TG2 expression is specific to transformed ovarian epithelial cells (p-value=0.005), but weak staining was observed in surface epithelial inclusions (FIG. 1B). Control staining (without primary antibody) was consistently negative.

### TG2 is Secreted in EOC Malignant Ascites.

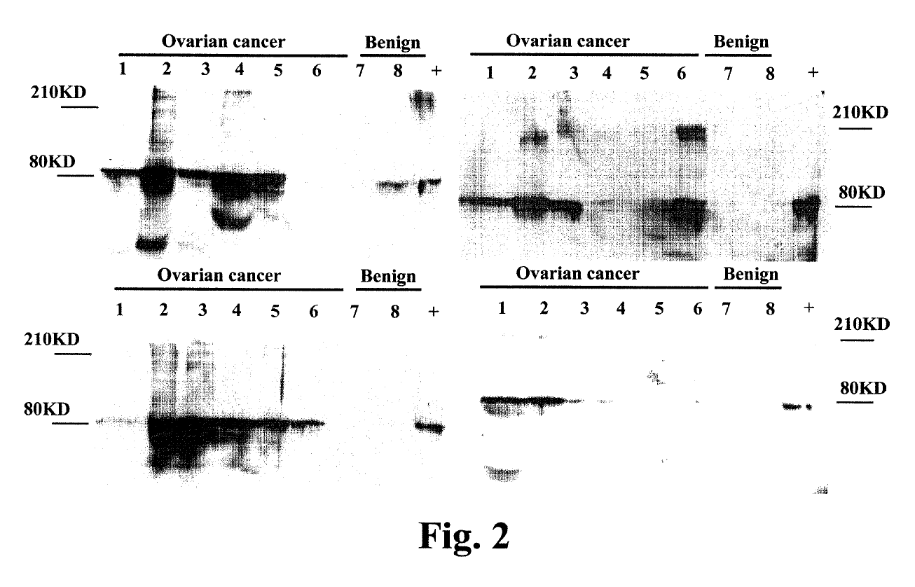

Because ovarian cancer disseminates in the peritoneal cavity, large volumes of ascites are generated, containing proteins secreted by tumor and mesothelial cells. Such secreted proteins modulate the growth and spread of carcinoma cells in the peritoneal milieu. TG2 is a secreted protein, we tested ascites whether TG2 is detectable in ascites. Immunoblot analysis revealed the presence of TG2 in 25 of 30 ascites in specimens from patients with EOC (representative immunoblots illustrated in FIG. 2). Eight specimens of non-malignant, inflammatory effusions assayed included negligible amounts of TG2, suggesting that TG2 secretion is specific to cancer cells (p-value=0.005). Immunoblot analysis revealed a higher molecular weight band, migrating at ˜170 kD in several ascites specimens. This was also observed in conditioned media from some of the ovarian cancer cell lines (not shown) and was disrupted by stringent denaturing conditions (SDS or 2-mercapthoethanol), suggesting that it represents a disulfide linked dimer. These unexpected results suggest that TG2 is up-regulated in EOC cells and secreted in malignant ascites in a cancer-specific manner.

### TG2 Facilitates Ovarian Cancer Cell Adhesion to FN.

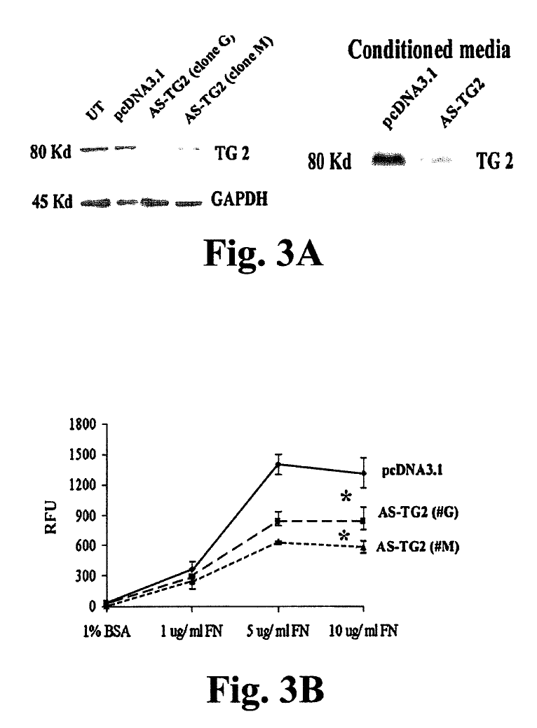

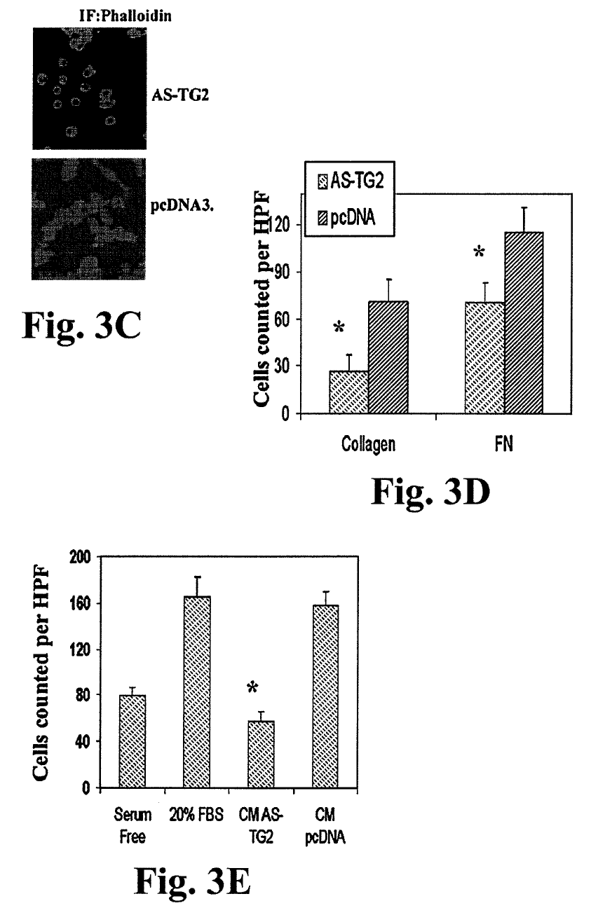

To try and determine the function of TG2 in ovarian cancer cells, we generated stable human cell lines, in which TG2 was either over-expressed or knocked down. To knock down TG2, we used an anti-sense construct (AS-TG2) cloned in pcDNA3.1 (25) in SKOV3 ovarian cancer cell line, which expresses abundant TG2. Two stable clones (G and M) were selected based on G418 resistance and screening by immunoblotting. Decreased TG2 level was noted in whole cell lysates and conditioned media from these two clones compared to vector transfected cells (FIG. 3A). Using a solid phase assay, we found that stable knock-down of TG2 in SKOV3 cells decreased ovarian cancer cell adhesion to FN by more than 50% at all concentrations of FN compared to control cells (FIG. 3B). Cell spread on FN was visualized by staining of the cytoskeleton with rhodamine-phalloidin. AS-TG2 transfected cells were round and failed to extend lamellipodia, whereas cells transfected with vector (pcDNA3) spread readily on FN (FIG. 3C). The effects on haptotactic cell migration were measured by a transwell assay. Collagen and FN-stimulated chemotaxis were decreased in SKOV3 cells stably transfected with AS-TG2 compared to cells transfected with vector (FIG. 3D). Additionally, conditioned media (CM) from control cells stimulated directional cell motility of SKOV3 cells. This was inhibited when the assay was performed with CM from AS-TG2 transfected cells (FIG. 3E).

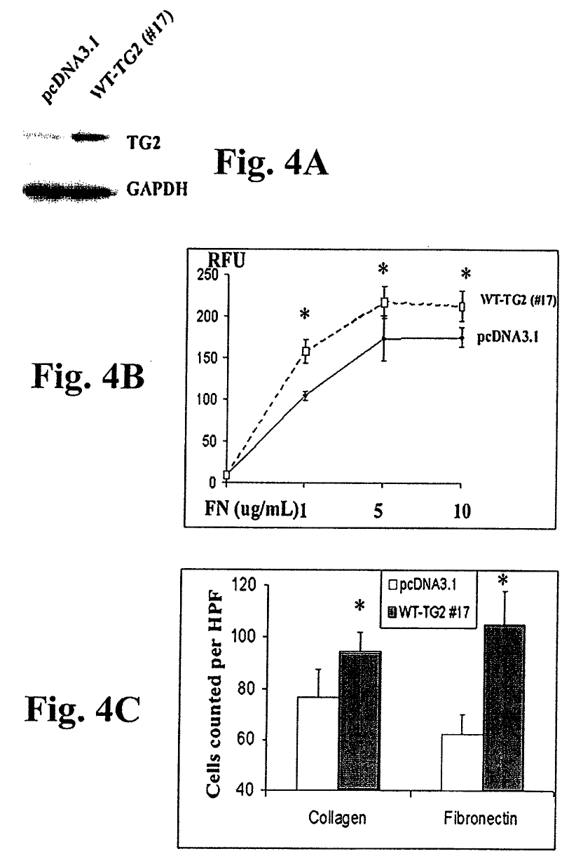

Next, we tested whether stable over-expression of TG2 increases adhesion to FN. For this, an ovarian cancer cell line with low endogenous level of TG2 (OV90) was transfected with TG2. A stably transfected clone was identified by immunoblotting after selection with G418 (FIG. 4A). Stable expression of TG2 enhanced adhesion to the FN compared to cells transfected with empty vector (FIG. 4B). Likewise, haptotactic cell migration stimulated by FN or collagen was enhanced by stable expression of TG2 (FIG. 4C). Coupled with the effects of TG2 knock-down, these experiments show that TG2 is critical to ovarian cancer cell adhesion and directional migration, essential steps in metastasis.

### TG2 Knock-Down Inhibits Tumor Development and Spread on the Peritoneal Surface In-Vivo

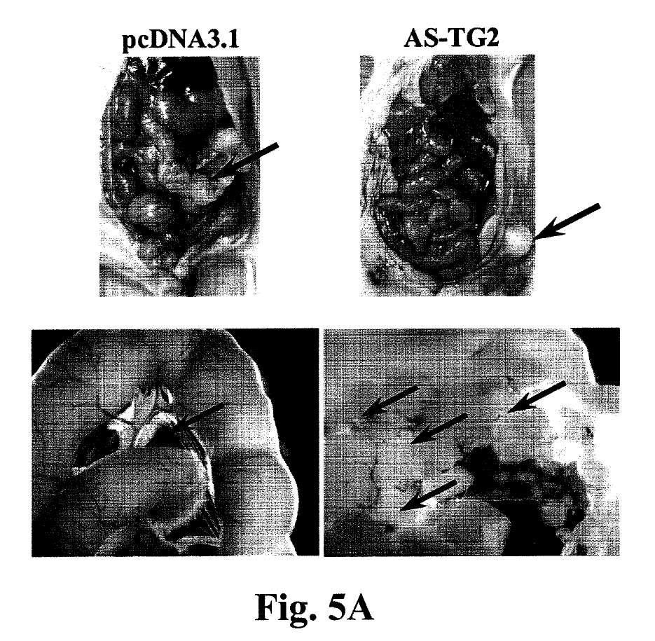

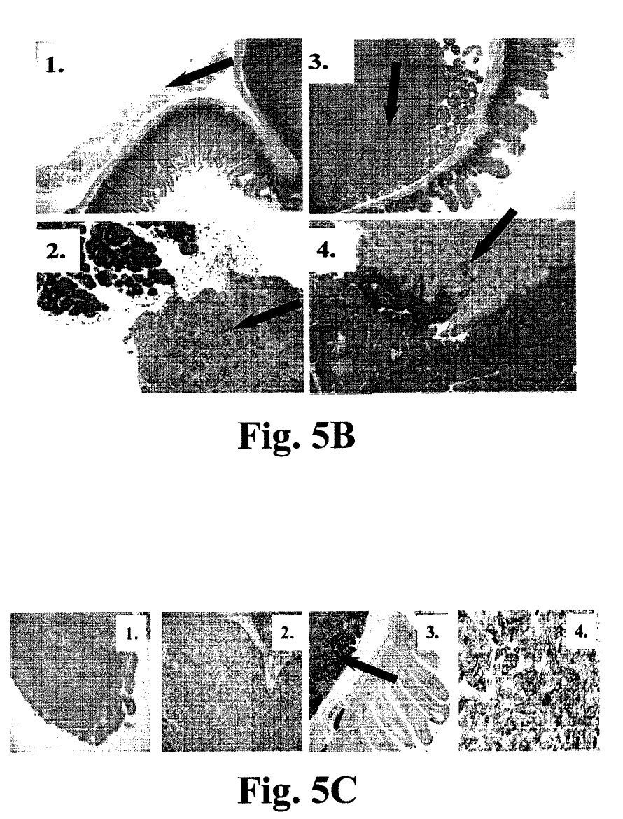

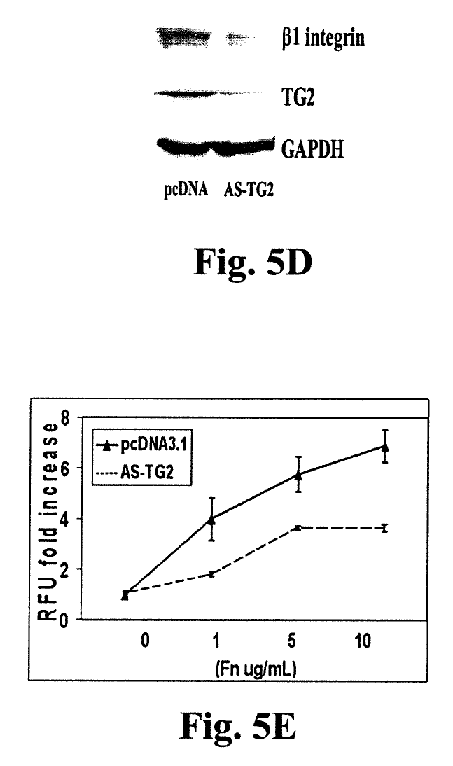

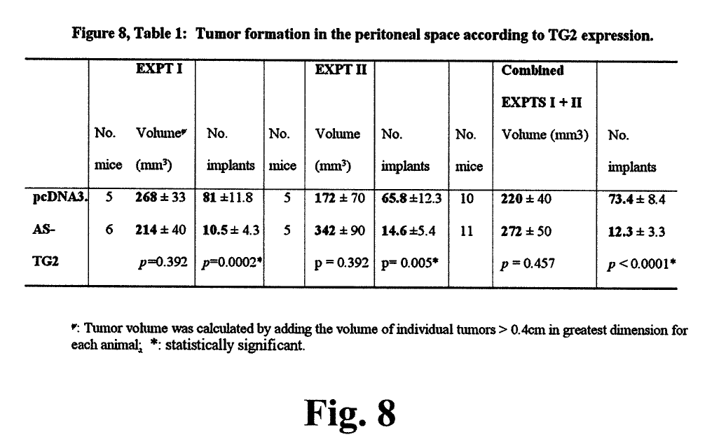

We injected nude mice i.p. with SKOV3 cells stably transfected with AS-TG2 (SEQ ID No. 2) or empty vector and after 8 weeks we observed a significant difference in pattern of tumor development. Mice injected with pcDNA3.1 transfected cells developed tumors in the omentum and the retroperitoneal (RP) space and numerous 1-3 mm tumors studding the mesentery, adjacent to the bowel and on the peritoneal surface of abdominal flanks (FIGS. 5 A and B) The average number of implants was 73+/−8. Mice injected with AS-TG2 transfected cells developed one large tumor in the omentum, invading into the RP space and a tumor nodule at the injection site, but significantly fewer mesenteric implants (12.3+7-3, FIG. 8 Table 1). The tumor volume of dominant masses was not different for AS-TG2 derived xenografts compared to controls, although a trend in favor of AS-TG2 xenografts was noted (Table 1). Tumors were of similar histological appearance, with high nuclear grade for both groups and a serous papillary pattern was discernable in mesenteric implants. TG2 knock down was preserved in-vivo, as demonstrated by IHC in xenografts and by Western blot analysis of cell cultures established from explanted xenografts (FIGS. 5 C and D). Occasional islands of TG2 positive cells were observed in tumors derived from AS-TG2 cells, consistent with the emergence of TG2 positive subpopulations in the absence of G418 selection in-vivo. Consistent with preserved TG2 knock-down in-vivo, cell cultures derived from explanted xenografts conserved their original phenotype; decreased adhesion to FN being noted between cells cultured from AS-TG2 and from control tumors (FIG. 5E). The pattern of tumor formation in the peritoneal space was consistent with the phenotype observed in-vitro, suggesting an important role for TG2 in the process of peritoneal seeding. This may be the first time that transglutaminase has been linked to metastasis and this unique observation provides a potential mechanism that can be targeted for treatment of ovarian cancer.

TG2 Interacts with β1 Integrin and Modulates its Expression

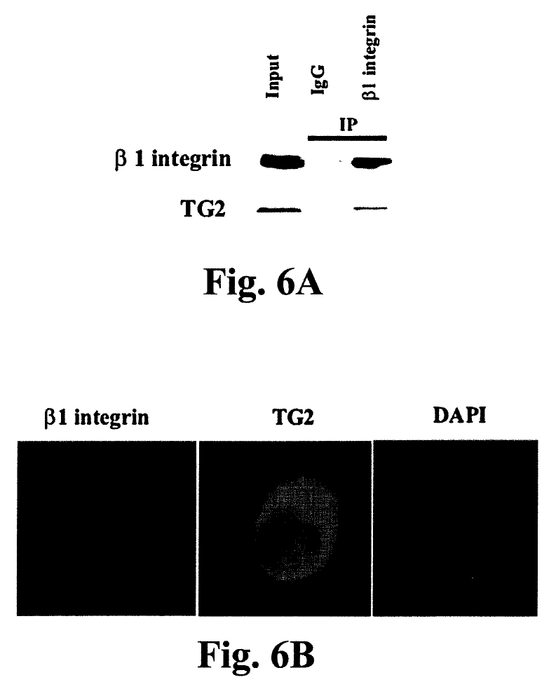

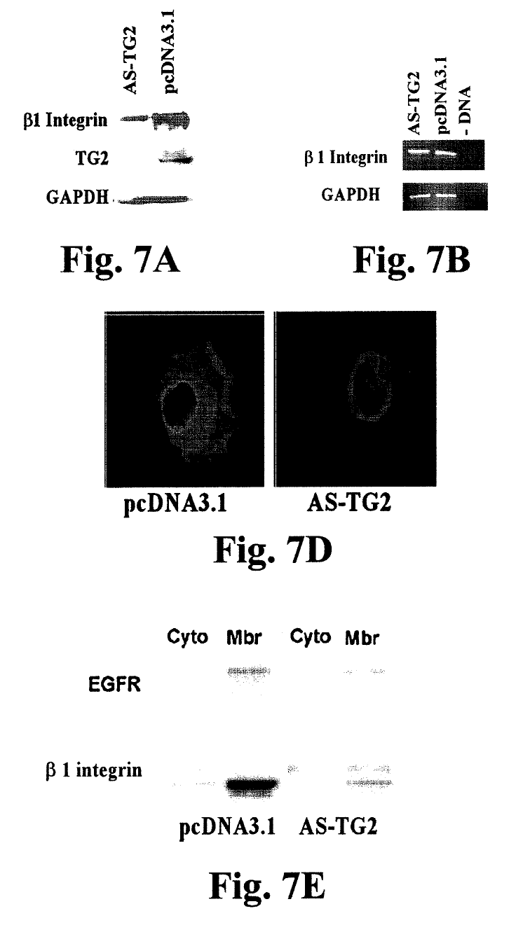

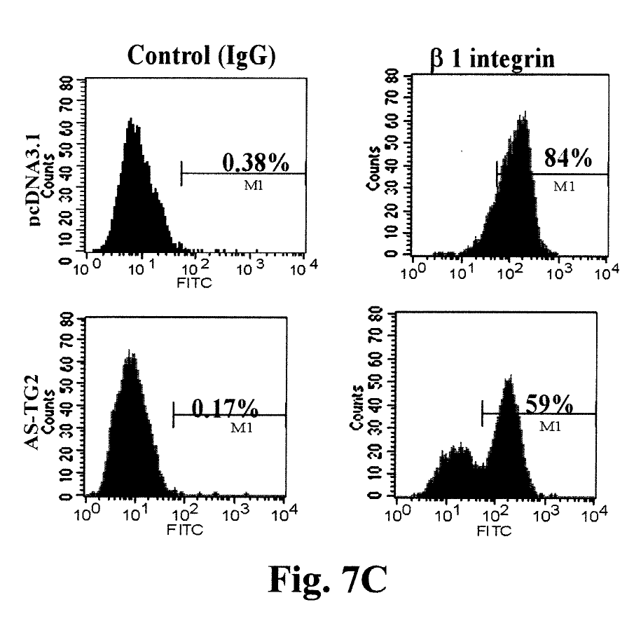

As alteration in the level of TG2 expression modulates EOC cell adhesion in-vitro and in-vivo, we examined whether TG2 interacts with integrins. Immunoprecipitation with β1 integrin antibody followed by immunoblotting for TG2 demonstrates endogenous interaction between TG2 and β1 integrin subunit in EOC cells (FIG. 6A). TG2 and β1 integrin co-localize in the cytoplasm and on the inner aspect of the cell membrane, suggesting that a functional complex is formed (FIG. 6B). To understand the relevance of TG2-β1 integrin interaction, we examined the level of integrin in cells with diminished TG2 expression and found that β1 subunit is expressed at decreased levels in AS-TG2 transfected cells (FIG. 7A). However, mRNA levels are not different compared to control cells (FIG. 7B), suggesting that TG2 affects integrin processing post-transcriptionally. Expression of β1 integrin on the cell surface was estimated by FACS and immunoblotting of membrane fractions. Decreased levels of β1 subunit at the plasma membrane were observed in SKOV3 transfected with AS-TG2 compared to controls (FIGS. 7C and E). Similarly, immunofluorescence demonstrated localization of β1 subunit in the peri-nuclear space in AS-TG2 cells, with reduced distribution of the integrin to the cell membrane (FIG. 7D). This suggests that TG2 affects β1 integrin's expression and function, at the spreading edges of the cancer cell, supporting its role in cell motility, adhesion and ultimately, in peritoneal seeding.

One embodiment includes treating or administering to a patient in need thereof a therapeutically effective dose of a compound that alters TG2 activity, the compound, or a pharmaceutically acceptable salt thereof, may be selected from the group consisting of phenylthiourea-(CH₂)ₙ—NH₂ wherein, n is =to 2, 3, 4, 5 and. For additional information on the synthesis, and characterization of these compounds please see, Lee, K. N., et al., JBC Vol. 260, No. 27 Iss. Nov. 25, pp. 14689-14694 (1985).

One embodiment includes treating or administering to a patient in need thereof a therapeutically effective dose of a compound that alters TG2 activity, the compound, or a pharmaceutically acceptable salt thereof, may be selected from the group consisting of: N-benzyloxy carbonyl, 5-deazo-4-oxonorvaline p-nitrophenylester, glycine methyl ester, CuSO₄, tolbutamide, monodanzyl cadaverine, putrescine, a monoamine, a diamine, gamma-amino benzoic acid, and derivates thereof. For additional information on the synthesis and characterization of these compounds please see, U.S. Pat. No. 6,794,414 issued on Sep. 21, 2004 to Steinman, which is incorporated herein by reference in its entirety.

One embodiment includes treating or administering to a patient in need thereof a therapeutically effective dose of a compound that alters TG2 activity, the compound, or a pharmaceutically acceptable salt thereof, of cystamine. For additional information on the synthesis and characterization of this cystamine please see, Zorniak, M., Eukaryon, Vol. 2, January 2006.

One embodiment includes treating or administering to a patient in need thereof a therapeutically effective dose of a compound that alters TG2 activity, the compound, or a pharmaceutically acceptable salt thereof of formula 1:

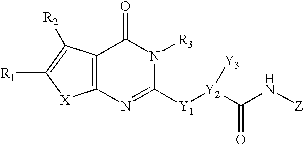

wherein,
R₁ is selected from the group consisting of: H, halogen, alkyl, substituted alkyl, aryl and substituted aryl;
R₂ is selected from the group consisting of: H, alkyl, substituted alkyl, aryl and substituted aryl;
R₃ is selected from the group consisting of: alkyl, substituted alkyl, aryl, substituted aryl, pyridine and substituted pyridine;
X is selected from the group consisting of: S, O and NH;
Y₁ is selected from the group consisting of: S, CH₂, NH, O and N-alkyl;
Y₂ is selected from the group consisting of: CH, alkyl and substituted alkyl;
Y₃ is selected from the group consisting of: H and CH₃;
Z is selected from the group consisting of: OH and NH₂;
with the provision that

when X is S, Yᵢ is S, Y₂ is CH, Y₃ is H, Z is NH₂, R₁ is Me, and R₂ is Me,

R₃ can not be Ph or a propylene group (CH₂═CH—CH₂—); and

When X is S, Y₁ is S, Y₂ is CH, Y₃ is H, Z is NH₂, R₁ is H, and R₂ is Ph, R₃ can not be Ph.

In still another embodiment, the compound according to formula 1 comprises the following groups:

R₁ is selected from the group consisting of: H, Me and Cl;

R₂ is selected from the group consisting of: phenyl and substituted phenyl;

R₃ is selected from the group consisting of: phenyl and substituted phenyl;

X is S;

Y₁ is S;

Y₂ is CH;

Y₃ is H; and

Z is NH₂.

One embodiment includes treating or administering to a patient in need thereof a therapeutically effective dose of a compound that alters TG2 activity, the compound, or a pharmaceutically acceptable salt thereof of formula 2:

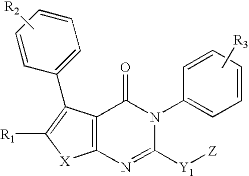

wherein,
R₁ is selected from the group consisting of: H, halogen and Me;
R₂ is selected from the group consisting of: H, 4-F, and 2-F;
R₃ is selected from the group consisting of: H, 4-F, and 3-F;
X is selected from the group consisting of: S, O and NH;
Y₁ is selected from the group consisting of: S, O, NH and NMe; and
Z is selected from the group consisting of: CH₂C(O)NHNH₂, CH₂CH₂C(O)NHNH₂, CH(Me-) C(O)NHNH₂, CH₂C(O)NMeNH₂, CH₂C(O)NHNHMe, CH₂CO₂H, CH₂CO₂Et, CH₂C(O)NHMe, CH₂C(O)NH₂ CH₂C(O)NHOH, and CH₂C(O)CH₂NH₂.

One embodiment includes treating or administering to a patient in need thereof a therapeutically effective dose of a compound that alters TG2 activity, the compound, or a pharmaceutically acceptable salt thereof of formula 3:

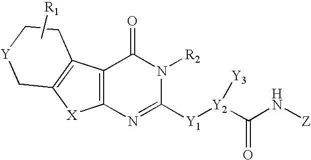

wherein,
Y is selected from the group consisting of: CH₂, N-Boc, NH, NMe, N-alkyl; and
R₁ is selected from the group consisting of: H, Me, Ph, alkyl, arylalkyl, t-butyl, and CH₂Ph;
R₂ is selected from the group consisting of: alkyl, substituted alkyl, aryl, substituted aryl; pyridine, and substituted pyridine;
X is selected from the group consisting of: S, O and NH;
Y₁ is selected from the group consisting of: S, CH₂, O, NH, N-alkyl;
Y₂ is selected from the group consisting of: CH, alkyl and substituted alkyl;
Y₃ is selected from the group consisting of: H and CH₃; and
Z is selected from the group consisting of: OH and NH₂.

For additional information on the synthesis and characterization of compounds 1, 2 and 3 please see, U.S. Patent Publication No. 2006/0183795, to Stein, et al., published on issued on Aug. 17, 2006, which is incorporated herein by reference in its entirety.

One embodiment includes treating or administering to a patient in need thereof a therapeutically effective dose of a compound that alters TG2 activity, the compound, or a pharmaceutically acceptable salt thereof of formula 4:

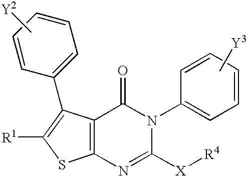

wherein,
R₁ is selected from the group consisting of: H and Me;
Y₂ is selected from the group consisting of: H, 4-F, and 2-F;
Y₃ is selected from the group consisting of: H, 4-F, and 3-F;
X is selected from the group consisting of: S, O, NH, and NMe; and
R₄ is selected from the group consisting of: CH₂C(O)NHNH₂, CH₂CH₂C(O)NHNH₂, CH(Me)C(O)NHNH₂, CH₂C(O)NMeNH₂, CH₂C(O)NHNHMe, CH₂CO₂H, CH₂CO₂Et, CH₂C(O)NHMe, CH₂C(O)NH₂, CH₂C(O)NHOH, and CH₂C(O)CH₂NH₂.

One embodiment includes treating or administering to a patient in need thereof a therapeutically effective dose of a compound that alters TG2 activity, the compound, or a pharmaceutically acceptable salt thereof of formula 5:

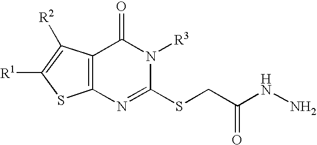

wherein,
R₁ is selected from the group consisting of H, Cl, Me, iPr, and Ph;
R₂ is selected from the group consisting of: Ph, Me, i-Pr, 4-OMe-Ph, 3-OMe-Ph, 2-OMe-Ph, 2-OH-Ph, 2-(OC₃H₆—NEt₂)-Ph, 4-F-Ph, 3-F-Ph, 2-F-Ph, and H; and
R₃ is selected from the group consisting of: Ph, Me, CH₂Ph, 3-Py, Cy, 2-OMe-Ph, 3-OMe-Ph, 4-OMe-Ph, 2-Cl-Ph, 3-CL-Ph, 4-Cl-Ph, 2-F-Ph, 3-F-Ph, and 4-F-Ph.

One embodiment includes treating or administering to a patient in need thereof a therapeutically effective dose of a compound that alters TG2 activity, the compound, or a pharmaceutically acceptable salt thereof of formula 6:

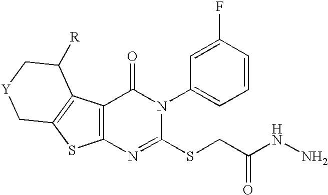

wherein,
Y is selected from the group consisting of: CH₂, N-Boc, NH, NMe, and N-n-Pr; and
R is selected from the group consisting of H, Me, Ph, CH₂Ph, and H.

For additional information on the synthesis, and characterization of compounds 4, 5 and 6 please see, Duval, E., et al., “Structure-Activity Relationship Study of Novel Tissue Transglutaminase Inhibitors,” Bioorganic and Medicinal Chem. Let., 15, pp. 1885-1889, (2005).

While the invention has been illustrated and described in detail in the figures and foregoing description, the same is to be considered as illustrative and not restrictive in character, it being understood that only the preferred embodiments have been shown and described and that all changes and modifications that come within the spirit of the invention are desired to be protected. As well, while the invention was illustrated using specific examples, theoretical arguments, accounts, and illustrations, these illustrations and the accompanying discussion should by no means be interpreted as limiting the invention. All patents, patent applications, and references to texts, scientific treatises, publications, and the like referenced in this application are incorporated herein by reference in their entirety.

We claim:

1. A method of assessing ovarian cancer peritoneal metastasis, comprising the steps of:
obtaining a sample of material from a patient; and
assaying at least a portion of said sample for elevated levels of Transglutaminase-2.

2. The method according to claim 1, wherein said sample is a portion of tissue.

3. The method according to claim 1, wherein said sample is a portion of ascites fluid.

4. The method according to claim 1, wherein said sample is a portion of fluid recovered from the peritoneal space.

5. The method according to claim 1, wherein said assaying step includes
contacting at least a portion of said sample with at least one antibody, wherein the antibody preferentially binds with at least one isoform of Transglutaminase-2.

6. A method of treating peritoneal metastasis of ovarian cancer, comprising the steps of:
providing at least one compound that alters the level or activity of Transglutaminase-2, or a pharmaceutically acceptable salt thereof; and
administering a therapeutically effective dose of said compound to a patient.

7. The method according to claim 6, wherein said compound is an anti-sense molecule, wherein said anti-sense molecule interferes with the biosynthesis of at least one isoform of Transglutaminase-2.

8. The method according to claim 7, wherein said anti-sense molecule includes at least one sequence having at least 80 percent identity to Anti Sense-Transglutaminase-2.

9. The method according to claim 6, wherein said compound is selected from the group consisting of: cystamine, N-benzyloxy carbonyl, 5-deazo-4-oxonorvaline p-nitrophenylester, glycine methyl ester, CuSO₄, tolbutamide, monodanzyl cadaverine, putrescine, a monoamine, a diamine, gamma-amino benzoic acid, and derivates thereof.

10. The method according to claim 6, wherein said wherein said compound is selected from the group consisting of: phenylthiourea-(CH₂)ₙ—NH₂, wherein, n is =to 2, 3, 4, 5

11. The method according to claim 6, wherein said compound is formula 1: 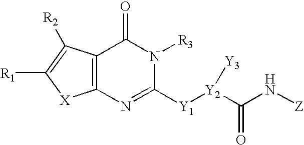
wherein,
R₁ is selected from the group consisting of: H, halogen, alkyl, substituted alkyl, aryl and substituted aryl;
R₂ is selected from the group consisting of: H, alkyl, substituted alkyl, aryl and substituted aryl;
R₃ is selected from the group consisting of: alkyl, substituted alkyl, aryl, substituted aryl, pyridine and substituted pyridine;
X is selected from the group consisting of: S, O and NH;
Y₁ is selected from the group consisting of: S, CH₂, NH, O and N-alkyl;
Y₂ is selected from the group consisting of CH, alkyl and substituted alkyl;
Y₃ is selected from the group consisting of: H and CH₃;
Z is selected from the group consisting of: OH and NH₂;
with the provision that
when X is S, Yᵢ is S, Y₂ is CH, Y₃ is H, Z is NH₂, R₁ is Me, and R₂ is Me,
R₃ can not be Ph or a propylene group (CH₂═CH—CH₂—); and
when X is S, Yᵢ is S, Y₂ is CH, Y₃ is H, Z is NH₂, R₁ is H, and R₂ is Ph, R₃ can not be Ph.

12. The method according to claim 11, wherein,
R₁ is selected from the group consisting of: H, Me and Cl;
R₂ is selected from the group consisting of: phenyl and substituted phenyl;
R₃ is selected from the group consisting of: phenyl and substituted phenyl;
X is S;
Y₁ is S;
Y₂ is CH;
Y₃ is H; and
Z is NH₂.

13. The method according to claim 6, wherein said compound is formula 2: 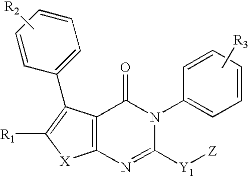
wherein,
R₁ is selected from the group consisting of: H, halogen and Me;
R₂ is selected from the group consisting of: H, 4-F, and 2-F;
R₃ is selected from the group consisting of: H, 4-F, and 3-F;
X is selected from the group consisting of: S, O and NH;
Y₁ is selected from the group consisting of: S, O, NH and NMe; and
Z is selected from the group consisting of: CH₂C(O)NHNH₂, CH₂CH₂C(O)NHNH₂, CH(Me-) C(O)NHNH₂, CH₂C(O)NMeNH₂, CH₂C(O)NHNHMe, CH₂CO₂H, CH₂CO₂Et, CH₂C(O)NHMe, CH₂C(O)NH₂ CH₂C(O)NHOH, and CH₂C(O)CH₂NH₂.

14. The method according to claim 6, wherein said wherein said compound is formula 3: 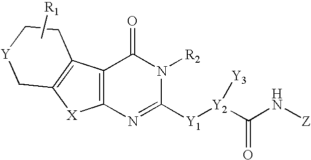
wherein,
Y is selected from the group consisting of: CH₂, N-Boc, NH, NMe, N-alkyl; and
R₁ is selected from the group consisting of: H, Me, Ph, alkyl, arylalkyl, t-butyl, and CH₂Ph;
R₂ is selected from the group consisting of: alkyl, substituted alkyl, aryl, substituted aryl;
pyridine, and substituted pyridine;
X is selected from the group consisting of: S, O and NH;
Y₁ is selected from the group consisting of: S, CH₂, O, NH, N-alkyl;
Y₂ is selected from the group consisting of: CH, alkyl and substituted alkyl;
Y₃ is selected from the group consisting of: H and CH₃; and
Z is selected from the group consisting of: OH and NH₂.

15. The method according to claim 6, wherein said compound is formula 4: 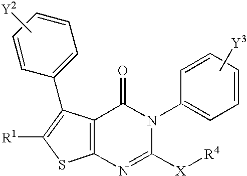
wherein,
R₁ is selected from the group consisting of: H and Me;
Y₂ is selected from the group consisting of: H, 4-F, and 2-F;
Y₃ is selected from the group consisting of: H, 4-F, and 3-F;
X is selected from the group consisting of: S, O, NH, and NMe; and
R₄ is selected from the group consisting of: CH₂C(O)NHNH₂, CH₂CH₂C(O)NHNH₂, CH(Me)C(O)NHNH₂, CH₂C(O)NMeNH₂, CH₂C(O)NHNHMe, CH₂CO₂H, CH₂CO₂Et, CH₂C(O)NHMe, CH₂C(O)NH₂, CH₂C(O)NHOH, and CH₂C(O)CH₂NH₂.

16. The method according to claim 6, wherein said compound is formula 5: 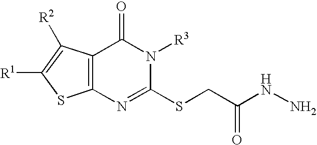
wherein,
R₁ is selected from the group consisting of: H, Cl, Me, iPr, and Ph;
R₂ is selected from the group consisting of: Ph, Me, i-Pr, 4-OMe-Ph, 3-OMe-Ph, 2-OMe-Ph, 2-OH-Ph, 2-(OC₃H₆—NEt₂)-Ph, 4-F-Ph, 3-F-Ph, 2-F-Ph, and H; and
R₃ is selected from the group consisting of: Ph, Me, CH₂Ph, 3-Py, Cy, 2-OMe-Ph, 3-OMe-Ph, 4-OMe-Ph, 2-Cl-Ph, 3-CL-Ph, 4-Cl-Ph, 2-F-Ph, 3-F-Ph, and 4-F-Ph.

17. The method according to claim 6, wherein said compound is formula 6: 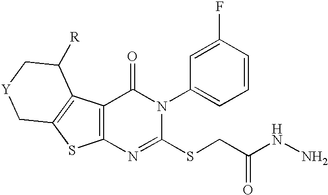
wherein,
Y is selected from the group consisting of: CH₂, N-Boc, NH, NMe, and N-n-Pr; and
R is selected from the group consisting of: H, Me, Ph, CH₂Ph, and H.

18. A method of regulating ovarian cell adhesion, comprising the steps of:
modulating the level of at least one isoform of Transglutaminase-2 in a ovarian cancer cell.


SEQUENCE LISTING (5 sequences)

SEQ ID NO: 1 - protein, 687 aa, Homo sapiens
MAEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPA

SEQ ID NO: 2 - DNA, 20 bp, Artificial Sequence
  B1 integrin primer F
atctgcgagtgtggtgtctg

SEQ ID NO: 3 - DNA, 20 bp, Artificial Sequence
  B1 integrin primer R
acaacatgaaccatgacctc

SEQ ID NO: 4 - DNA, 22 bp, Artificial Sequence
  GAPDH primer F
gattccacccatggcaaattcc

SEQ ID NO: 5 - DNA, 18 bp, Artificial Sequence
  GAPDH primer F
cacgttggcagtggggac